(19) United States
(12) Patent Application Publication
(10) Pub. No.: US 2007/0270923 A1
(43) Pub. Date: Nov. 22, 2007

(54) Incising Cell to Basement Membrane Bonds

(76) Inventor: Rajeev Raut, Pune (IN)

Correspondence Address: BROOKS KUSHMAN P.C., 1000 TOWN CENTER, TWENTY-SECOND FLOOR, SOUTHFIELD, MI, 48075, US

(21) Appl. No.: 11/574,111

(22) PCT Filed: Dec. 24, 2004
(86) PCT No.: PCT/IN04/00410

§ 371 Date: Feb. 22, 2007

(30) Foreign Application Priority Data
Aug. 23, 2004 (IN) 905/MUM/2004

(51) Int. Cl. A61N 5/06

(52) U.S. Cl. 607/88

(57) ABSTRACT

Cells are attached to each other and to a basement membrane, to form a layer or layers. Cells may be separated from basement membrane without damaging the cells or basement membrane by the devices disclosed here. The devices enable simultaneous exposure of the cell basement membrane complex to light energy from both sides, the cells side and the basement membrane side. This simultaneous exposure of the cell basement membrane complex layer to specific levels of light energy from two sides causes incision of the bonds that attach the cells to the basement membrane.

### FIELD OF INVENTION

The present invention discloses a device to incise bonds between cells and basement membrane, without damaging the cell or the basement membrane. The device enables exposure of the cell basement membrane complex to specific intensity light energy from two directions, with the light energy incident on the cell side of is of very low intensity, and the light energy incident on the basement membrane side is of higher intensity, to achieve the incision of the bonds between them.

The effects of light on cell basement membrane complex depend on wavelength, intensity, duration of exposure, inherent composition of the tissue at the time of exposure, and on direction in which the exposure is affected. The application deals with achieving specific incision of the cell to basement membrane bonds by using very low intensity light exposure from the cells side, simultaneous higher intensity exposure from the basement membrane side, with light energy of specific wavelengths.

### BACKGROUND OF THE INVENTION

In laboratory procedures and in various surgical procedures, it is necessary to effectively isolate cells which adhere to basement membranes or capsules due to various reasons. Such isolation has to be effective to prevent further complications or deterioration of the membrane or tissues, facilitate better visualisation of structures or tissue behind the cells or basement membranes, and to achieve optical advantages like for example, better staining for photographing the cells, and better manipulation for studying their properties.

There are several devices and methods as disclosed in the prior art, which aim to separate cells which adhere to the cell membranes.

The present invention relates to a device and a method, which overcomes the various problems associated with prior Art. The invention embodies devices emitting light of selected wavelengths of low intensity for separating epithelial cells. The device enables an operator to expose the cell basement membrane complex to light energy from two directions, to achieve the desired effect of isolating epithelial cells from the basement membrane to which they are attached. The effect is achieved by incising the bonds between cell and basement membrane.

The device can be employed in a number of therapeutic, laboratory and scientific procedures.

In the human body and in the laboratory, one comes across many situations where the cells are lined up on a basement membrane in a single layer or in many layers. For example, in the human eye, on the corneal surface, the epithelium is arranged on a basement membrane called the Bowman's membrane, in four to six orderly layers. The attachments between the cells and the basement membrane is very strong. These epithelial cells ar very resistant to light that comes onto them from outside. However, we have found from our research, that the attachments of these cells to basement membrane are very fragile and vulnerable to light energy, if the light is directed onto these attachements from the inner side, at low intensity when at the same time, stronger light falls on the cells from the outer side.

In a mammal, lens epithelial cells of eye proliferate after the rest of the lens material is removed during cataract surgery. They may become opaque, and cause “after cataract” which affects vision. Some of these cells change their character after surgery, become fibroblasts, and may cause fibrous scar formation in the capsule, giving rise to capsule contraction syndrome. Even if the cells do not produce any of these problems, they cause opacification of the capsule, and hinder visualisation of the structures posterior to it. This makes treatment and examination of the retina very difficult, for optical reasons.

It is desirable to remove these cells during cataract surgery to avoid all these problems in the postoperative period.

The cell membrane such as eye capsule is very thin and fragile. The space in which the surgeon has to work is very limited, and the capsule must be spared along with the surrounding tissue, at all cost. The inner structures of the eye do not tolerate any high-energy insults like chemicals, heat, electricity, laser, mechanical abrasions, etc.

The lens epithelial cells are attached to the capsule, from inside. They do not come out by simple washing as the attachment between the cells and the capsule is very strong. If this attachment is loosened or severed, the cells can be washed out easily, or may be sucked out by a simple tubular irrigating cannula attached to a syringe.

These cells can not be ablated by a laser device, because the cells will then die and the dead cells will stick to the capsule, causing optical problems in the post operative period.

The prior Art in the field discloses various means for overcoming the problem of removing the epithelial cells.

Some of the prior art discloses use of mechanical means to remove unwanted cells. The chief limitation of these methods is the possibility of injury to the surrounding tissue.

International Patent Publication WO 00/49976, PCT/US00/04339 describes a Nicapsulorhexis Valve. This is a silastic valve which will attach to the capsulorhexis opening, in a water tight fashion. This excludes the rest of the inner surface of the eye from contact with certain cytotoxic substances, which may be introduced into the capsular bag, to destroy the epithelial cells.

International Patent Publication WO 99/04729, deals with an Intraocular Ring as a device. This disclosure deals with a physical gadget called intra ocular ring, which kills the cells or prevents their proliferation, by He pressure effect caused by its contact with the cells.

International Patent Publication WO 2004/039295 describes a method of making a capsulorhexis in the lens capsule. The lens is removed from the lens capsule of an eye and the capsulorhexis is sealed with a sealing means/device, to provide gas leakage proof sealing. The lens capsule is expanded with a gas and desired operation is performed inside the said expanded lens capsule.

Here, the inventor discloses a air tight sealing device seals the capsular bag from the rest of the eye so that toxic gases or liquids may be introduced into the bag to kill the cells.

U.S. Pat. No. 6,432,078 describes a System and Method for removing cataract or other cells in an eye using water jet and a suction. It discloses a mechanical device to abrase, and then to suck the cells out of the eye, using water jet, mechanical brushes, etc.

International Patent Publication WO 98/25610/PCT/CA97/00949, discloses use of green porphyrins for the manufacture of a medicament for the treatment of secondary cataracts. In this document, researchers from the University of Columbia disclose certain chemical substances called green porphyrins. These chemical substances are applied to the epithelial cells, and then irradiated with light, so that they destroy the cells to which the substance is applied. This has called photodynamic therapy of the lens capsule.

Porphyrins are chemical substances, which must be introduced into the eye. The method is therefore not desirable.

International Patent Publication WO 99/39722, PCT/IB99/00905 discloses compositions and methods for separating lens epithelial cells and preventing posterior capsular opacification This is achieved by modulating focal contacts, which mediate adhesion between lens epithelial cells and the lens capsule, using a treating solution containing a focal contact-modulating substance or a proenzyme, such as Lys-plasminogen, which is introduced into the eye.

International Patent Publication WO 02/047728, PCT/GB01/05465 discloses treatment of posterior capsule opacification. This disclosure deals with killing the cells with a chemical ligand. The ligand is preferably Fas ligand. A spacer is preferably polyethylene glycol. The polymer preferably constitutes an intraocular lens.

International Patent Publication WO 02/43632, PCT/AU01/01554 discloses a device for sealing the capsular bag of an eye and a method for delivering fluid or treatment substances to the lens of an eye. A method is disclosed to seal the capsular bag from the rest of the eye, at the same time allowing delivery of strong chemicals into the bag, to kill the cells.

U.S. Pat. No. 4,966,577 discloses a composition for preventing secondary cataract formation in the eye following removal of the lens, comprising an antibody specific to particular lens cells related to secondary cataract formation, which antibody is conjugated to an antiproliferative agent. The particularly preferred antiproliferative agents require activation after binding of the antibody to the target cells, and activation may be accomplished by addition of a second composition or by exposure of the eye to electromagnetic energy. Also disclosed is a method of using the composition by administering it directly to the site from which the lens was removed to kill or prevent proliferation of lens cells.

This disclosure again specifies first, introduction of a chemical substance, then introduction of another chemical substance, and then activation of this combination by use of electromagnetic energy, to destroy the cells of the capsule. U.S. patents U.S. Pat. No. 5,620,013 U.S. Pat. No. 5,843,893, U.S. Pat. No. 5,627,162 disclose chemical agents to destroy the cells of the capsule.

The chief limitation to chemical methods disclosed above is toxicity and adverse effects of the chemicals to the surrounding tissue.

International Patent Publication WO 01/54603, PCT/US01/03052 discloses a system and method for treating cells of a site in the body, such as at a lens capsule of an eye. The system and method employs an energy emitting device, and a positioning device, adapted to position the energy emitting device at a position in relation to the cells at the site in the body, such as the cells of the lens capsule, such that energy emitted from the energy emitting device heats the cells to a temperature which is above body temperature and below a temperature at which protein denaturation occurs in the cells, to kill the cells or impede multiplication of the cells. The energy emitting device can include a container containing a heated fluid which heats the cells to the desired temperature. The disclosures here deal with a method that heats the cells to denature or coagulate them, thereby destroying them.

International Patent Publication WO 98/18392, PCT/US96/17322 discloses an instrument for destroying residual lens epithelial cells in a lens capsule of an eye. The said instrument comprising of an electrical energy source, a probe comprising an electrode, electrically coupled to said electrical energy source, and the said probe having a distal end portion configured for insertion into said eye between an iris of said eye and said lens capsule; and an insulating sleeve In this disclosure, the inventor discloses a method to electrically cauterise the capsule cells, so as to kill them.

The chief limitation of electrical methods is that the delicate tissue around the cells may also get cauterised

U.S. Pat. No. 6,669,694 discloses medical instruments and techniques for highly-localized thermally-mediated therapies. It describes delivery of high thermal energy to the tissue to achieve an ablative effect on the cells.

U.S. Pat. No. 4,963,142 discloses an apparatus for endolaser microsurgery. A method and apparatus for performing endolaser microsurgery is disclosed, the apparatus including a laser delivery system coupled to a probe capable of transmitting the laser energy through a suitable medium such as sapphire. The probe includes a coaxial canal for aspiration of ablated tissue and/or fluids. The method involves steps of ablating tissue by laser and aspirating the ablated tissue and/or fluids, the method being useful for sclerostomy, vitrectomy and as a substitute for ultrasonic phacoemulsification among others. A probe for performing endolaser microsurgery and removing ablated tissues is described. The apparatus disclosed here is meant to deliver laser energy, and to ablate the tissue, followed by removing the ablated tissue.

The term ablation, is a geological term. By definition, it means “melting away” or removal away by melting or evaporation. The laser energy described here is a means to achieve a high energy level, high enough to melt the tissue, and then to remove the ablated or melted products. The achievement of high energy is done by using laser, which allows very high energy concentration at a small area, for a short time, and achieves the melting with out damaging the surrounding tissue.

U.S. Pat. Nos. 6,238,386, 6,554,824, 6,582,421, 6,712,808, 6,726,680 disclose an instrument that applies laser energy to human tissue.

U.S. Pat. No. 6,454,762 discloses an instrument for applying light, especially laser light, to the human or animal body. It describes an instrument which consists of a movable tip, which enables light energy or laser energy from an external source to be directed to the desired part of the human body.

U.S. Pat. No. 6,238,386 discloses application of sound energy and laser energy to internal body cavities by endoscope. The application of energy inside the human body by fiber optic delivery system. The laser used is therapeutic laser and supplies laser radiation at an optical power at said distal end which is at least 5 Watts or at an intensity at said distal end which is at least 1 kW cm.sup.-2. The power is disclosed to be such as is required for coagulating tissue.

Muller discloses a device for using laser energy and sound energy for treating inner body parts endoscopically, but the device uses energy, as stated above, to coagulate tissue. The minimum energy disclosed in the said invention is 5 watts. As 1 watt=408 lux ,the magnitude of energy used will be 2040 lux/cm.sup-2 or 2040,0000 lux/metersq.

The device disclosed in this application uses very low energy from the cell side, of a maximum of up to 1000 lux/sq mtr, simultaneously using higher energy from the basement membrane side. There is no coagulation at this energy levels. The device disclosed herein points the energy to the cells basement complex simultaneously in two specific directions, from cell side and from the basement membrane side, to achieve the desired effect.

The lasers involve high energy, and may cause thermal damage or thermal coagulation of the tissue by raising the temperature of the tissue to high levels for a fraction of a second. However, the surrounding tissue can also get ablated when high energy systems like lasers are used. Such energy will certainly damage the underlying capsule, if the epithelial cells were to be coagulated. It is well known that the capsule breaks at energy levels of 1.2 millijoules, therefore, the disclosed device in the said invention can not be used in ophthalmology to separate epithelial cells from the capsule. This damages the cornea and the capsule itself.

### LIMITATIONS OF PRIOR ART

The prior art cited above attempts to stop the problems associated with the capsular epithelial cells by destroying them and then removing the cells by the following general means:
A. Mechanical means These methods disclose mechanical devices for the removing the unwanted cells. The chief limitation of these methods is the possibility of injury to the surrounding tissue.
B. Chemical means. These methods use chemicals for removing the cells. The chief limitation to this method is toxicity of these chemicals to surrounding tissue.
C. Electrical means. The chief limitation is again, the delicate tissue around the cells may also get cauterised.
D. Laser or Sonic methods/bright light sources The lasers involve high energy, and achieves thermal damage or thermal coagulation of the tissue by raising the temperature of the tissue to high levels for a fraction of a second. However, the surrounding tissue can also get ablated when high energy systems like lasers are used. This damages the cornea and the capsule itself.
The objective of gently isolating the cells from basement membrane can not be achieved with a laser, because the photocoagulated cells stick to the basement membrane, and cause even stronger adhesion than before exposure to the laser.

### SUMMARY OF THE INVENTION

The invention embodies a device that affects exposure of cell basement membrane complex to specific low intensity light energy from one direction viz from the cell side and higher light energy from the basement membrane side simultaneously to affect isolation of these cells from the basement membrane. The device may embody fiber optic tips or delivery mirror, which enables the exposure of cells to the energy from two specific directions simultaneously. The present invention overcomes the various shortcomings of the prior art by providing a device embodying a low intensity light source and method/s to expose the epithelial cells in such manner as to loosen the attachment between the epithelial cells and the capsule. The removal of cells from the basement membrane may be carried out by simple washing, if desired.

This is achieved by directly exposing the target cells to a pre-selected very low intensity light of wavelengths between 194 to 850 nanometers on the cells side and simultaneously exposing the basement membrane side to higher intensity light energy, by a device and a method. The low intensity light is directed onto the cells from the cell side and not from the basement membrane side. The light is delivered to the cells from inside, by almost actually touching the tip of the light source carrier to the cell-capsule complex, and the distance between the epithelial cells and the light source is almost zero. The time of exposure is less than 60 seconds. The basement membrane side of the cell basement membrane complex is exposed to light energy of selected specific wavelengths between 194 nanometers and 850 nanometers. The light may be coherent or non coherent. The light that falls on the cell basement membrane complex specified here is from the 194 to 850 nanometers and the illuminance specified here of 0.002 to 5,00 000 lux.

The cells are extremely resistant to this light, if it comes from the normal outer side, but extremely sensitive to this light if it is directed to them from the inner side in the manner provided in the invention. The basement membrane side of the cell basement membrane complex must be exposed to a higher intensity light energy of the illuminance from 0.002 lux to 500000 lux.

### BRIEF DESCRIPTION OF THE DRAWINGS

FIG. 1 is a diagrammatic representation of low intensity device for separating epithelial cells.

1 Light source for exposing the basement membrane side of the cell basement membrane complex

2 Light source 2 for exposing the cells side of the cell basement membrane complex.

3 Basement membrane

4 cells

FIG. 2 shows the device using a single external light, where a filter and attenuator regulate the intensity and wavelength of the light falling on the cell basement membrane complex from the basement membrane side and from the cell side, so that the exposure from the cell side is of very low intensity compared to the exposure from the basement membrane side. The light is being carried by fiber optic cables.

1 Single light source

5 Fiber optic cable

6 Filter and attenuator and polariser to carry the light energy to the basement membrane side of the complex.

7 Filter and attenuator and polariser to carry dimmer light of different specific spectral composition to the cell side of the cell basement membane complex.

FIG. 3 Shows an external light, which falls on the basement membrane directly, but is directed onto the cell side be a reflecting mirror. The attenuators, filters and polarisers are depicted in a schematic manner, and shall be obvious to those skilled in the art. The exposure should be such that the energy falling on the basement membrane side of the cell basement membrane complex is higher than that falling on the cell side of the cell basement membrane complex.

1 light source wavelength 194 to 1600 nanometers.

9 filter/polariser/attenuator

3 Basement membrane

4 cells

10 filters polarisers/attenuators

8 mirror

FIG. 4 Shows the exposure of the basement membrane from a light source from outside, which passes through the transparent cornea, and exposes the outer side of the lens capsule to the light energy, whereas a fiber optic carries light from another source, or the same source, but modified by filters and attenuators and exposes the cell side of the complex to light energy. 11 external light source such as an aperating microscope light source

12 light passing from the external light source onto the basement membrane side of the tissue.

13 Fiber optic carrying the light energy from another light source or from the same light source, but attenuated and filtered, onto the other side of the cell basement membrane complex, ie from the cells side.

14 The inner side or the cell side of the cell basement membrane complex is being exposed to the light carried there by the fiber optic, with a smooth atraumatic tip.

15 Cornea, which is transparent.

16 cut portion of the capsular bag called capsulorhexis opening.

17 Outer side of the capsular bag.

FIG. 5 shows smooth tip either in contact or close to the capsule From inside. and shows curved tip, dual source device, with correct method of exposure and placement of the device tip.

19,18 fiber optic light sources

16 capsule or basement membrane

17 cells lining the capsule from inside.

FIG. 6 shows two smooth curved hooks, made of fiber optic cords or encasing fiber optic cords. The smooth hooks are autraumatic, and this is done to avoid injury to other biological structures which may be close. The distance of the light carrier to the cells side of the cell—basement membrane complex is very dose to the cells.

20 Exposure to the basement membrane side of the basement membrane-cell complex by an atraumatic design smooth curved light transport system

21 basement membrane aspect being exposed.

22 Second light source exposing the cells side of the cell basement membrane complex by another smooth surfaced atraumatic cannula

23 Cells side of the cell basement membrane complex.

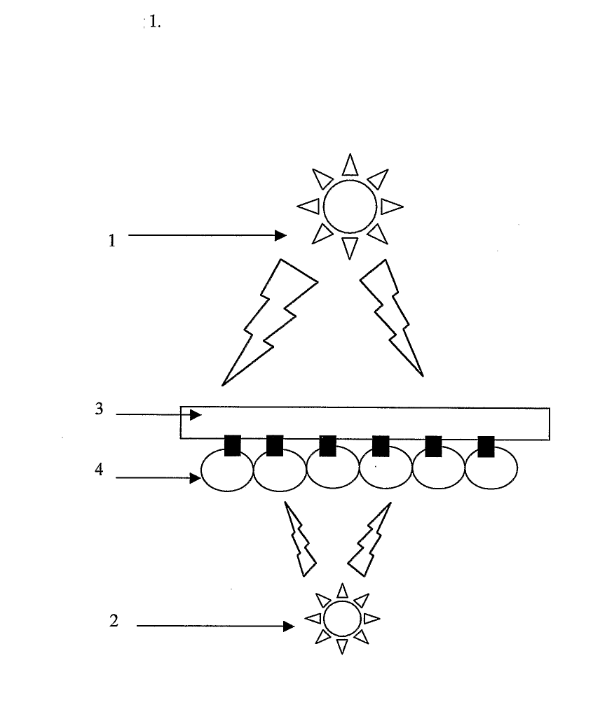

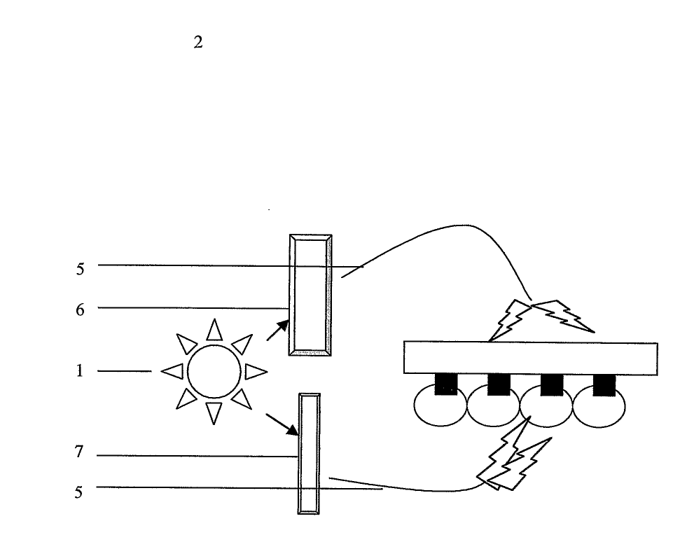

The invention will now be described with reference to the FIGS. 1 to 6 described above.

The device for incising cell basement membrane bonds consists of a light source (1), and a transport system (5,13,17,18) to carry this light into the specific site, and if the basement membrane or capsule is shaped like a curled bag or an envelope, to carry the energy into the capsular bag, through the opening into the bag. The tip of the transport system (14,20,22) where the instrument comes in contact with the capsule is smooth, and atraumatic.

In another embodiment, two light pipes carry light into the eye, one goes into the inside of the capsular bag, and the other illuminates the capsular bag from outside, as shown in sheet 5, labelled as parts 19, 18.

Light Source

The light source may be coherent or non coherent, monochromatic or multichromatic. It may be a LED, or may be laser source, arc lamp source, tungsten filament, light source, or any other light source day light may be used and modified as a light source.

The light source may be white, or may be of colors. A white light source may be converted into a source of certain pure colors by using filters. Single light source with filters may be used to create pure color wavelengths and the inside of the capsule bag may be exposed to pure colors. A Mixed light source of white light may also be used. Wavelength selected is between 194 to 850 nanometers.

Intensity is the critical part of the device. The intensity of the light source used in the invention must be such that the final incident light which falls on the cells must be of very low intensity to produce illuminance of 0.001 lux to about 1000 lux. It may be noted that a 40 watts domestic light bulb produces illuminance of thousands of lux if measured very close to the surface of the bulb.

The light source may be switched or pulsed on and off several times a second, in one of the preferred embodiments.

The light source may be more than one, so that cells are exposed to different wavelengths of light, alternately.

In combination with the first light source, there must be a second light source used. This may be an external light source of the surgical microscope, or a totally different light source, by which, light is carried onto the basement membrane. Such a light source may be a tiny LED, daylight which is modified by filters, optical focussing lenses, or polarisers or attenuators, laser light source, external bulb light source.

In one of the preferred embodiments, such a light source is used with an illuminance of 0.002 lux to 500000 lux.

This second light source is mandatory and it must illuminate the basement side of the cell basement membrane complex with illuminance higher than that of the illuminance of the first light source which works from inner or cell side of the cell basement membrane complex. The exposure should be simultaneous, to get the best effect. The second light source may be white, but may be of different colors.

To meet the condition that the energy incident on the basement membrane side is higher than that incident on the cells side, more than one light sources may be used, to expose the basement membrane side of the cell basement membrane complex.

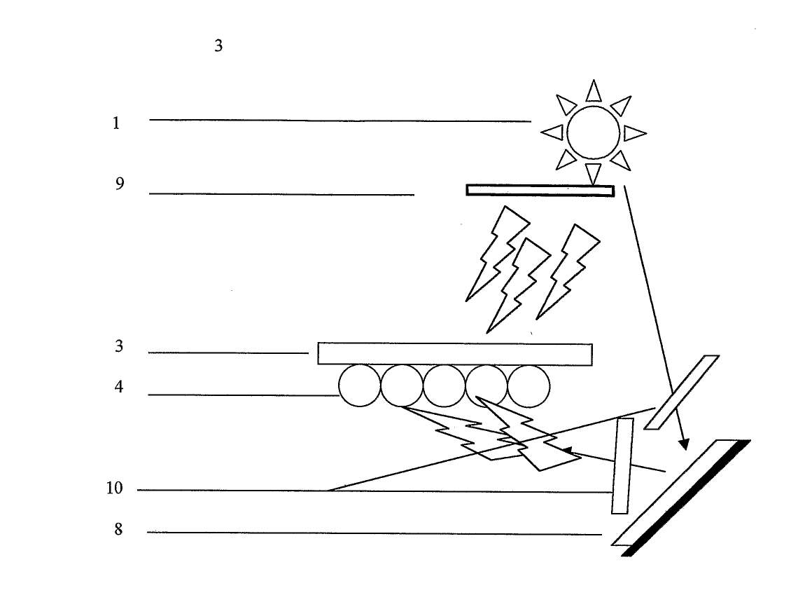

If the first light source is white light, and if filters are used to produce pure wavelengths to be delivered into the inside of the capsule, the first light source may be used also as the second light source, by bypassing the filters, and adding new filters and attenuators as shown in FIG. 2.

Delivery System

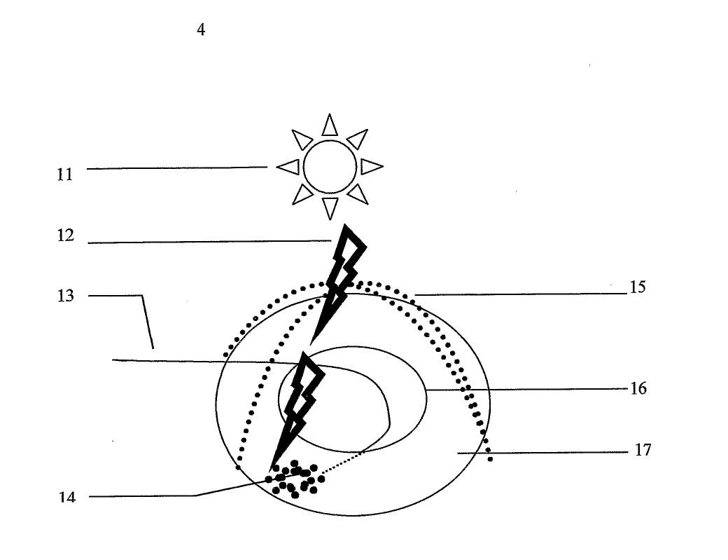

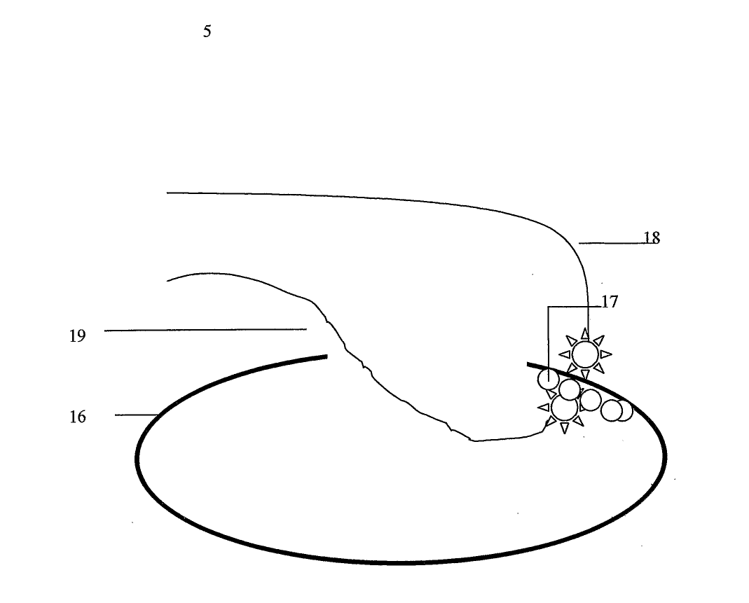

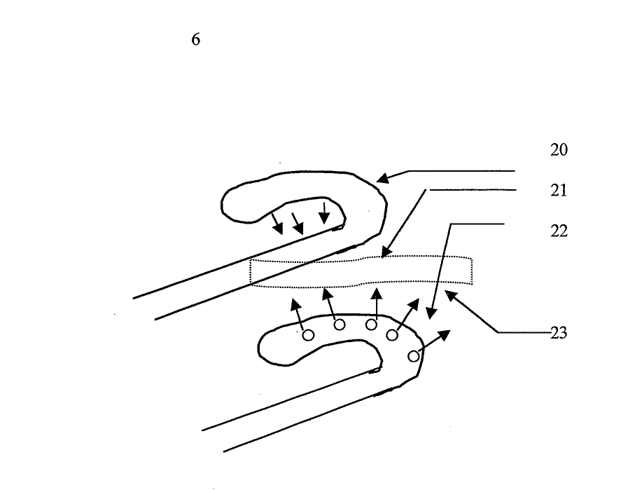

Fiber optic cable (5 in FIG. 2, 13 in FIG. 4, 18,19 in FIG. 5), or reflecting mirrors (8, in FIG. 3 ) are used to deliver the light energy to the cells directly. The fiber optic cable may be enclosed in a transparent water tight tubular cannula to avoid its contact with the tissues of the eye.

The tip of the cannula (14 in FIG. 4 and 20,22 in FIG. 6) is smooth, rounded, so that when it comes in contact with the under surface of the capsule, it does not tear or damage it.

Method

During the actual procedure, after first, all debri and dirt that may be stuck to the cell basement membrane complex is cleaned by gentle suction and wash. If the procedure is being carried out in a laboratory, in a dish or a container, the liquid in which the cell basement membrane complex is stored is kept free from dirt or insoluble floating particles. When the procedure is used inside the human body, like during cataract surgery, the nucleus of the cataract is removed. The cortex is cleaned. The low intensity light is carried through the device into the capsular bag, and the cells are exposed to it from inside. The microscope lamp may be used as a second light source for exposure from the basement membrane side. In a laboratory, the cell basement membrane complex may be placed on a slide and exposed from both sides to the light energy, with the energy from low intensity source falling directly on the cells side. In the laboratory, when the procedure is performed under a microscope, the microscope lamp may be used as the second bright source, which will expose the basement membrane side to the higher energy simultaneously. The cells are freed/separated by the exposure of cell surface to low intensity and basement membrane surface to high intensity light from the device. The isolated epithelial cells can be removed if desired by known methods such as simple wash and suction.

The device is effective by exposing the capsule cells to light from both sides at the same time. One beam of light falls on the anterior capsule from outside. This beam is either from the source of light used by the surgeon as an operating microscope, or a source of light located outside, and brought on to the anterior surface of the capsule by a light pipe made of fiberoptic. The light which falls on the anterior capsule from outside may be of an illuminance from 0.002 lux to 500000 lux.

However this outer beam of light alone does not form the device, the device must essentially contain the inner beam of light which falls simultaneously onto the cells from inside, with specified low illumination.

The light from the source which is used to treat the cells from inside the capsule may be turned on and off one to fifteen times a second.

In another embodiment of the invention the light energy is transported to the inside of the anterior capsule by a set of mirrors placed in a bent pipe, so that instead of a fiber optic carrier, the light travels through the hollow pipe and is turned into required path by these reflecting mirrors and prisms.

In another embodiment of the invention the light source is directly carried to the point where exposure of capsule cells is possible without passing this light through fiber optic cable, by the use of reflecting mirrors as shown in FIG. 3.

The present invention, however, is not limited to any particular application or environment. Instead, those skilled in the art will find that the present invention may be advantageously applied to any application or environment using different low intensity light sources or combinations in multiple thereof, methods for applying such low intensity light sources by any other direct or indirect methods or means, the use of mirrors or any other reflecting device. The description of the exemplary embodiments, which follows, is therefore, for the purpose of illustration and not limitation.

### Most Preferred Embodiment

### A. The Device

Two light sources, one comsisting of blue and red LEDs where the blue light is 360 to 420 nanometers, and the red LED is from 700 to 850 nanometers. The LEDs are pulsed from zero times a second to fifteen times a second. This light source is used to expose the cell basement membrane complex from inner or cell surface. The intensity is very low, so that illuminance on the cell surface is 0.001 to 1000 lux.

The second light source is the light directly used from a surgical microscope. This light is used to illuminate the basement membrane side of the cell basement membrane complex, directly, through the cornea. To facilitate exposure, the pupil is dilated by eye drops or mechanically by the surgeon, so that the iris moves out of the way of the second light source. The intensity used is such that the illuminance of the basement membrane is 0.002 to 5,00,000 lux.

The light coming out of the first light source is picked up by a fiber optic light pipe, which carries it to the inside of the eye.

The end piece of the fiber optic is a cannula (20,22 in FIG. 6) whose tip is transparent, and allows this light to be delivered to the capsule.

### B. Preferred Embodiment—Method

For the application of the low intensity device for separating epithelial cells, the cannula is applied inside the capsular bag emptied of the nucleus and the cortex, and the second light from the operating microscope is allowed to fall on the basement membrane by either medically dilating the pupil preoperatively or by mechanically pulling the iris away, by the surgeon. The capsule is touched from inside, with the first cannula at many places, allowing the light from the device to fall momentarily on different regions of the capsule. Cells are loosened and may even already start floating in the fluid in the anterior chamber. These may be removed by known methods such as washing with gentle irrigation and aspiration, either with a hand held syringe and cannula, or with the automated system available with most phacoemulsification machines.

In its most preferred embodiment, this device is different from the mechanical devices disclosed in the prior art. The device of the invention does not contain any movable parts, does not transmit any high intensity light onto the cells, and/transmits light of only certain well defined wavelengths, for a well defined low intensity and for a well defined period of time, specifically to a well defined part of the cell basement membrane complex.

The device described in the application uses light energy, with specified energy levels on the cell side which are several thousand times lower than those used by prior art. The energy delivery in the invention does not aim to “coagulate” tissue, The device disclosed in this application uses very low light energy on the cell side and higher energy on the basement membrane side of the cell basement membrane complex to gently separate or loosen the cells, by incising the bonds between cell and basement membrane so that the cells can be isolated.

The typical laser energies used in the prior art disclose energies several thousand times more than the energy delivered as specified in this application. The device disclosed in the application uses illuminance levels of 0.001 lux to a maximum of 1000 lux from the cells side and simultaneously a higher illuminance levels of 0.002 lux to 500000 lux.

from the capsule side or the outer side. The energy required in the device disclosed herein is 0.0000024 watts for illumination from inside

1. A device for incising bonds between cells and basement membrane or capsule, comprising of a light source or sources, with specific wavelengths varying from 194 nanometers to 850 nanometers, with a transport system which simultaneously expose cell surface and basement membrane surface of the cell basement membrane complex to two different levels of light energy with the energy exposure on the cell side of the complex being very low in intensity, and an optical carrying system that carries this light energy so that the epithelial cells lining the capsule or basement membrane are exposed to the light emanating from the device directly without passing through the basement membrane so that one of them applies light energy from the device to the epithelial cells directly, without the energy having to pass through basement membrane.

2. The device of claim 1 where the light source that directly exposes the cells emits low light energy of a magnitude so low that the final available energy where it acts on the capsule epithelial cells, is between 0.001 lux and 1000 lux.

3. The device of claim 1 wherein the light source that acts on the cells side of the cell basement membrane complex is external and the light is carried to the capsule cells by a fiber optic pipe, which allows passage of light energy between 194 nanometers and 850 nanometers.

4. The device of claim 1 where the light source that acts on the cells side of the cell basement membrane complex is directly carried to the point where exposure of capsule cells is possible without passing this light through anterior or posterior capsules, by the use of reflecting mirrors.

5. The device of claim 1 where, if the basement membrane is like a bag or an envelop with the cells lining inside of the bag or envelop, the light energy is transported to the inside or cells side of the cell basement membrane complex by a set of mirrors placed in a bent pipe, so that instead of a fiber optic carrier, the light travels through the hollow pipe and is turned into required path by these reflecting mirrors and prisms.

6. The device of claim 1 where the light source itself is fitted into the hollow pipe so that it directly exposes the cells side of the cell basement membrane complex in the interior of the capsular bag.

7. The device of claim 1 where the additional light source which illuminates the capsular bag from outside, so that the capsular bag is illuminated from both inside and outside at the same time, is a coherent or non coherent light from an external source. Which is either monochromatic or polychromatic, with wavelengths between 194 to 850 nanometers. and with intensity such that the illuminance on the basement membrane is from 0.002 lux to 5,00,000 lux.

8. The device of claim 7 where the additional second light source is that of an operating microscope or any other external light.

9. The device of claim 7 where, if the basement membrane is shaped like a bag or an envelope, the second light source is carried by a fiber optic light source, directly onto the anterior or outer surface of the surface of the basement membrane or capsule.

10. The device of claim 7 where the second light source is carried onto the outer surface of the basement membrane or anterior capsule by using reflecting mirrors placed into the eye.

11. The device of claim 7 where the second light source is carried directly to the anterior or outer surface of the basement membrane placed over the membrane or capsule, to illuminate it directly.

12. The device of claim 1 where, if the basement membrane or capsule is shaped like a bag or an envelope, then the part of the device that goes into the capsular bag is smoothly turned into a round tip.

13. The device of claim 1 where the part of the device that goes into the capsular bag is turned into a round loop or sphere.

14. The method of using the device described in claim 1, by passing the tip of the device into the basement membrane, where the basement membrane or capsule is shaped like a bag or envelope, pointing it towards the capsular cells, and taking it very close to the cells, to touch them with the tip of the device, and after, washing or aspirating the cells out of the basement membrane complex by a conventional irrigation aspiration system.

15. The method of using the device of claim 1, where, if the basement membrane is shaped like a bag or an envelop, the first light exposes the cells from inside the bag, and the second light source illuminates the basement membrane or capsule capsule from outside, by placing the second tip on the outer or anterior surface of the basement membrane or capsule.

16. The device of claim 2 where the additional light source which illuminates the capsular bag from outside, so that the capsular bag is illuminated from both inside and outside at the same time, is a coherent or non coherent light from an external source, which is either monochromatic or polychromatic, with wavelengths between 194 to 850 nanometers and with intensity such that the illuminance on the basement membrane is from 0.002 lux to 5,00,000 lux.

17. The device of claim 3 where the additional light source which illuminates the capsular bag from outside, so that the capsular bag is illuminated from both inside and outside at the same time, is a coherent or non coherent light from an external source, which is either monochromatic or polychromatic, with wavelengths between 194 to 850 nanometers and with intensity such that the illuminance on the basement membrane is from 0.002 lux to 5,00,000 lux.

18. The device of claim 4 where the additional light source which illuminates the capsular bag from outside, so that the capsular bag is illuminated from both inside and outside at the same time, is a coherent or non coherent light from an external source, which is either monochromatic or polychromatic, with wavelengths between 194 to 850 nanometers and with intensity such that the illuminance on the basement membrane is from 0.002 lux to 5,00,000 lux.

19. The device of claim 5 where the additional light source which illuminates the capsular bag from outside, so that the capsular bag is illuminated from both inside and outside at the same time, is a coherent or non coherent light from an external source, which is either monochromatic or polychromatic, with wavelengths between 194 to 850 nanometers and with intensity such that the illuminance on the basement membrane is from 0.002 lux to 5,00,000 lux.

20. The device of claim 6 where the additional light source which illuminates the capsular bag from outside, so that the capsular bag is illuminated from both inside and outside at the same time, is a coherent or non coherent light from an external source, which is either monochromatic or polychromatic, with wavelengths between 194 to 850 nanometers and with intensity such that the illuminance on the basement membrane is from 0.002 lux to 5,00,000 lux.
